(19) United States
(12) Patent Application Publication
(10) Pub. No.: US 2012/0226287 A1
(43) Pub. Date: Sep. 6, 2012

(54) ENDOSCOPIC DEVICES FOR TISSUE REMOVAL OR REPAIR AND ASSOCIATED METHODS

(76) Inventor: Mohammed Abdul Qadeer, Lewisville, NC (US)

(21) Appl. No.: 13/410,556

(22) Filed: Mar. 2, 2012

Related U.S. Application Data
(63) Continuation-in-part of application No. 13/295,417, filed on Nov. 14, 2011.
(60) Provisional application No. 61/449,181, filed on Mar. 4, 2011.

(51) Int. Cl. A61B 17/26

(52) U.S. Cl. 606/113

(57) ABSTRACT

Endoscopic medical devices for removing tissue include: (a) a forceps with an elongate shaft slidably residing in a first working channel of an endoscope and having an externally accessible forceps control; and (b) a snare with an elongate shaft comprising a wire loop slidably residing in the first working channel of the endoscope, the snare having an externally accessible snare control. The forceps and snare shafts reside adjacent to and substantially parallel to each other inside the first working channel of the endoscope, one above or to the side of the other. In use, the forceps and snare cooperate so that the forceps control directs the forceps to extend out of the endoscopic first working channel and grasp target tissue, then the snare control directs the snare loop to exit the working channel while the snare loop encircles the forceps shaft and extends a distance sufficient to surround the forceps.

### RELATED APPLICATIONS

This application claims the benefit of and priority to U.S. Provisional Application Ser. No. 61/449,181, filed Mar. 4, 2011, and is a continuation in part of U.S. patent application Ser. No. 13/295,417 filed Nov. 14, 2011, the contents of these documents are hereby incorporated by reference as if recited in full herein.

### FIELD OF THE INVENTION

This invention relates to surgical devices that may be particularly useful for removing polyps or submucosal dissections during endoscopic procedures and/or tissue dissection and excision during NOTES.

### BACKGROUND OF THE INVENTION

Endoscopic procedures are well known. Natural orifices transluminal endoscopic surgery (NOTES) is a promising technology, but obstacles remain that inhibit the commercial use of the procedures in human patients including, for example, tissue dissection and excision. There remains a need for surgical tools for endoscopic surgeries.

### SUMMARY OF EMBODIMENTS OF THE INVENTION

Some embodiments of the invention are directed to endoscopic medical devices for removing tissue. The devices include: (a) a forceps with an elongate shaft slidably residing in a first working channel of an endoscope and having an externally accessible forceps control; and (b) a snare with an elongate shaft comprising a wire loop slidably residing in the first working channel of the endoscope, the snare having an externally accessible snare control. The forceps and snare shafts reside adjacent to and substantially parallel to each other inside the first working channel of the endoscope, one above or to the side of the other. In use, the forceps and snare cooperate so that the forceps control directs the forceps to extend out of the endoscopic first working channel and grasp target tissue, then the snare control directs the snare loop to exit the working channel while the snare loop encircles the forceps shaft and extends a distance sufficient to surround the forceps and grasped tissue so that the forceps and the snare loop cooperate while the snare excises the grasped target tissue.

The snare loop can be a closed loop that resides about the forceps shaft inside the working channel of the endoscope prior to extension from the working channel.

The snare loop can be configured to angulate with respect to the forceps to be able to take on different angles relative to the forceps.

The snare loop can be configured to move from an orientation that is substantially parallel to the forceps to an orientation that is between 30-60 degrees relative to a longitudinally extending centerline of the forceps.

The snare loop is configured to enlarge upon exiting the working channel.

Yet other embodiments are directed to methods of excising target tissue (e.g., a polyp). The methods include: (a) providing a medical tool having a forceps with an elongate shaft and a snare with an elongate shaft; (b) inserting the medical tool into a single working channel of an endoscope; (c) extending the forceps out of the working channel to grasp target tissue during an endoscopic procedure; then (d) extending the snare out of the working channel a distance while the snare loop surrounds the forceps shaft; then (e) surrounding the forceps and the grasped tissue with the snare loop; and (f) excising the target tissue using the snare loop while the forceps remains attached to the grasped target tissue.

The method may optionally also include angling the wire loop about the forceps shaft between about 30-60 degrees from an axially extending centerline of the forceps shaft during the extending step.

Other embodiments are directed to endoscopic tools. The tools include: (a) a forceps tool with an elongate shaft; and (b) a second tool with an elongate shaft. Each tool is configured to reside inside a single working channel of an endoscope.

The second tool can be a clip delivery device holding a deployable clip that attaches to local tissue.

The second tool can be at least one of a snare, secondary forceps, knob, scissors, knife, needle-knife or needle.

The forceps and second tool can be configured to cooperate for submucosal dissection during endoscopy and/or tissue dissection and excision during a NOTES procedure.

Yet other embodiments are directed to methods of removing polyps or a submucosal dissection. The methods include: (a) providing a medical device with a forceps and a second cooperating tool adapted to reside in a single working channel of an endoscope; (b) extending the forceps out of the working channel to grasp target tissue; then (c) using the second tool to carry out the submucosal dissection of the grasped tissue.

The second tool can include a wire snare.

The second tool can include at least one of a knife, a needle, a needle-knife or a knob.

Other embodiments are directed to methods of repairing a perforation during an endoscopic procedure. The methods include: (a) providing a medical device with a forceps and a second cooperating tool releasably holding a clip, both tools adapted to reside together in a single working channel of an endoscope; (b) extending the forceps out of the working channel to grasp target tissue to pull the target tissue to make a perforation smaller; then (c) using the second tool to attach the clip to close the perforation while the forceps holds the grasped tissue.

The foregoing and other objects and aspects of the present invention are explained in detail in the specification set forth below.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail in the specification set forth below.

### BRIEF DESCRIPTION OF THE DRAWINGS

FIGS. 1A-1C are schematic illustrations of an endoscopic surgical device according to embodiments of the invention.

FIGS. 2A and 2B are schematic illustrations of proximal (external) control units for the device shown in FIGS. 1A-1C according to embodiments of the present invention.

FIGS. 3A-3E are additional schematic illustrations of the device shown in FIGS. 1A-1C, illustrating snare angulation with respect to the forceps according to embodiments of the present invention

FIG. 4 is a partial view of the forceps shown in FIGS. 3A-3E with exemplary dimensional characteristics according to embodiments of the present invention.

FIGS. 5A-5C illustrate an exemplary series of steps that can be used to remove a polyp with the device shown in FIGS. 1A-1C, according to embodiments of the present invention.

FIGS. 5D-5F illustrate an exemplary series of steps for removing flat polyps using the device shown in FIGS. 1A-1C, according to embodiments of the present invention.

FIGS. 6A-6D illustrate an exemplary series of steps for removing polyps using the device shown in FIGS. 1A-1C to tent the target tissue and a provide cooperating alignment of the snare using the forceps as an anchor and/or guideline according to embodiments of the invention.

FIGS. 7A-7G are schematic illustrations of different embodiments of devices with various alternative tools useable with a primary forceps according to embodiments of the invention.

FIGS. 8A-8C illustrate a series of steps that can be used to close an aperture in a wall of a lumen using a clip delivery tool with a forceps according to embodiments of the present invention.

FIGS. 9A-9E illustrate a series of steps that can be used for submucosal dissection with a device including a primary forceps and a cooperating separate tool according to embodiments of the present invention.

FIG. 10 is a flow chart of exemplary method steps that can be carried out according to embodiments of the present invention.

FIG. 11 is a schematic illustration of a clip suitable for deployment from a channel of an endoscope according to embodiments of the present invention.

### DESCRIPTION OF EMBODIMENTS OF THE INVENTION

The present invention will now be described more fully hereinafter with reference to the accompanying figures, in which embodiments of the invention are shown. This invention may, however, be embodied in many different forms and should not be construed as limited to the embodiments set forth herein. Like numbers refer to like elements throughout. In the figures, certain layers, components or features may be exaggerated for clarity, and broken lines illustrate optional features or operations unless specified otherwise. In addition, the sequence of operations (or steps) is not limited to the order presented in the figures and/or claims unless specifically indicated otherwise. In the drawings, the thickness of lines, layers, features, components and/or regions may be exaggerated for clarity and broken lines illustrate optional features or operations, unless specified otherwise.

The terminology used herein is for the purpose of describing particular embodiments only and is not intended to be limiting of the invention. As used herein, the singular forms, “a”, “an” and “the” are intended to include the plural forms as well, unless the context clearly indicates otherwise. It will be further understood that the terms “comprises,” “comprising,” “includes,” and/or “including” when used in this specification, specify the presence of stated features, regions, steps, operations, elements, and/or components, but do not preclude the presence or addition of one or more other features, regions, steps, operations, elements, components, and/or groups thereof.

It will be understood that when a feature, such as a layer, region or substrate, is referred to as being “on” another feature or element, it can be directly on the other feature or element or intervening features and/or elements may also be present. In contrast, when an element is referred to as being “directly on” another feature or element, there are no intervening elements present. It will also be understood that, when a feature or element is referred to as being “connected”, “attached” or “coupled” to another feature or element, it can be directly connected, attached or coupled to the other element or intervening elements may be present. In contrast, when a feature or element is referred to as being “directly connected”, “directly attached” or “directly coupled” to another element, there are no intervening elements present. Although described or shown with respect to one embodiment, the features so described or shown can apply to other embodiments.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein.

Embodiments of the invention are useful for veterinarian and human uses as well as for animal studies. That is, methods and devices provided by embodiments of the invention can be configured for any species of interest, e.g., mammalian including human, simian, mouse, rat, lagomorph, bovine, ovine, caprine, porcine, equine, feline, canine, and the like.

Embodiments of the invention may be particularly suitable for removing polyps during an endoscopic procedure, including, for example, an endoscopic polypectomy, submucosal dissection of flat polyps, and flat mucosa associated with certain conditions such as Barrett's esophagus. For the latter, embodiments of the surgical devices can remove relatively large areas of mucosal tissue which are not otherwise amenable to endoscopy therapy at this time. It is also contemplated that devices according to some embodiments may be helpful in creating submucosal tunnels for identification of specific nerves or group of neurons, such as for a gastric pacemaker. Particular embodiments are directed to NOTES procedures in human patients including, for example, tissue dissection and/or excision, and potentially safe closure of an opening.

Generally stated, embodiments of the invention are directed to endoscopic devices with at least two tools that can work independently of each other and move relative to the other, but that synergistically allow for surgical procedures, such as, for example, polyp removal.

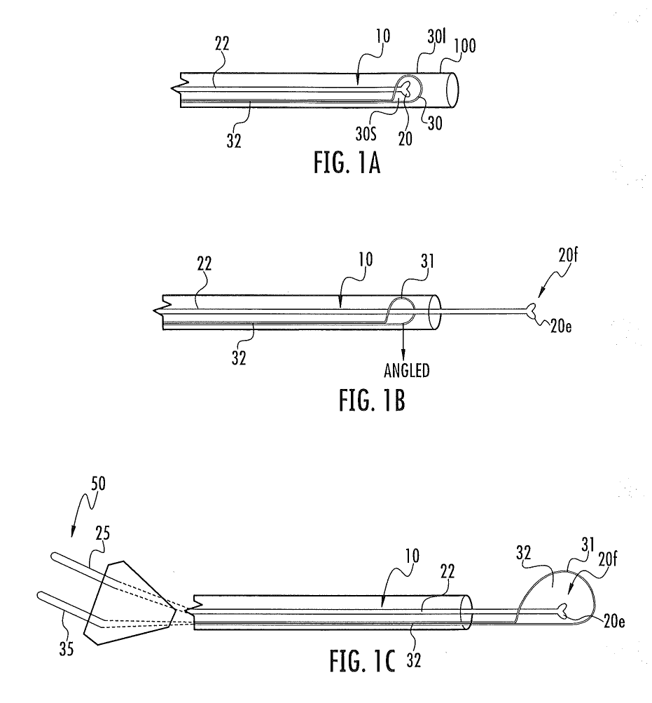

Referring now to FIGS. 1A-1C, the surgical device 10 includes at least two cooperating tools 20, 30 that reside in a single working channel 100 of an endoscope. As shown, one of the tools 20 is a forceps 20f. The end of the forceps 20e exits the working channel 100 to grasp and pull target tissue as will be discussed further below. The tool 20 has an elongate shaft 22 that connects to a control unit, typically a hand control unit 25 (FIG. 1C).

The forceps 20f can be deployed first to grab and pull or push tissue to provide traction so that the other tool 30 can be used to snare the tissue, inject the tissue, cut or dissect the tissue, control bleeding such as by placing a closure clip, or the like. Thus, the second (or more) tool 30 can be a snare, a secondary forceps, a needle, a needle-knife, a knob, scissors, or a delivery tool that places a clip to close a hole in tissue. Once the endoscope is in a desired position in the body, the primary forceps 20f can be deployed from the endoscope. Then, once the primary forceps 20f is in position attached to target tissue, the second tool 30 can be deployed from the working channel. The second tool 30 can perform the desired action on the grasped tissue, e.g., one or more of tissue dissection, cutting, cauterization, snaring, injection, and the like.

The depths of the at least two independently operable, but cooperating, tools 20, 30 can be selectively and individually varied. By varying the depth of the two tools 20, 30, and allowing the tools to cooperate, the primary forceps 20f can apply traction, and dissection and/or excision of the tissue can be accomplished through endoscopy as the device 10 is able to provide an element of triangulation.

The at least two tools 20, 30 can be provided in a catheter, tube, collar or sleeve (not shown) that resides in the working channel 100 or they may be used in the working channel without such a catheter, tube, collar or sleeve.

In some embodiments, the other cooperating tool 30, as shown in FIGS. 1A-1C, is a snare 30s with a wire loop 31. The loop 31 is shown as a closed loop but it may alternatively be configured as an open loop. The snare wire loop 31 can have any appropriate shape, e.g., an oval snare, a hexagonal snare, or a semicircular snare, and the snare may be a barbed snare or a hooked snare.

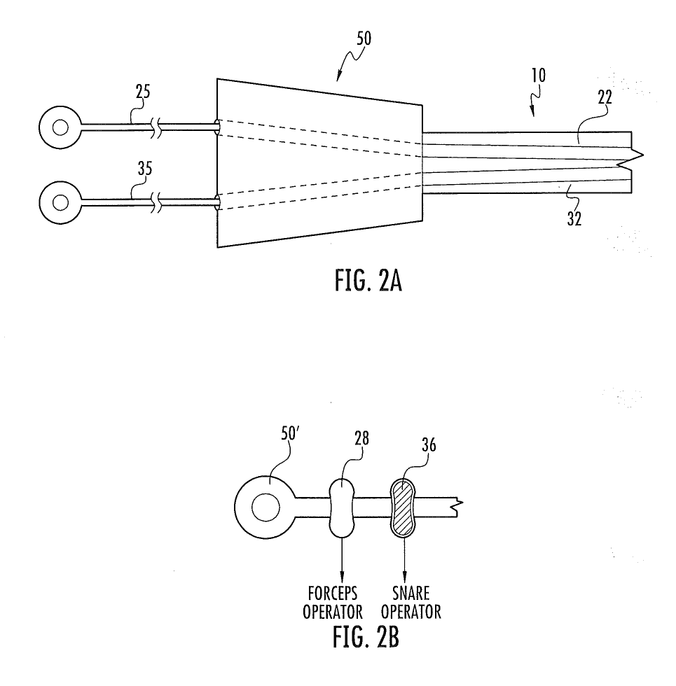

The tool 30 includes an elongate shaft 32. The elongate shaft 32 can connect to a control unit 50, such as an individual hand control unit 35 (FIGS. 1C and 2A). The shafts of the tools 22, 32 can reside side-by-side or one above another in a substantially parallel orientation.

The forceps 20f can extend through the loop 31 of the snare before, during or after it exits the working channel 100. The snare loop 31 can be angled upward between about 20-90 degrees, typically between about 30-60 degrees, from an axially extending center line of the shaft 20s and/or shaft 30s. That is, the snare loop can angle upward with respect to the forceps and define an angle associated with a line drawn through a centerline of a distal end of the snare shaft relative to a line drawn through a center or end of the loop.

The control units 25, 35 are configured to direct the forceps 20f and snare to exit the working channel 100. FIG. 2B illustrates a different control unit 50′ with an integrated shaft that can advance and retract the forceps 20f and snare 30s, but with individual rotational control elements 26, 36 that can triangulate the orientation of the respective tool 20, 30. The control units can be manually operated or, in some embodiments, may be robotically controlled.

As shown in FIGS. 1A and 1B, the forceps 20f can extend through a cutting loop 31 of the snare 30s. In operation, in some embodiments, the forceps 20f resides through the open space 32 defined by the interior of the loop 31 to grasp target tissue, e.g., even a flat polyp, and tent it so that the cutting loop 31 of the snare can excise the polyp off of the tissue wall.

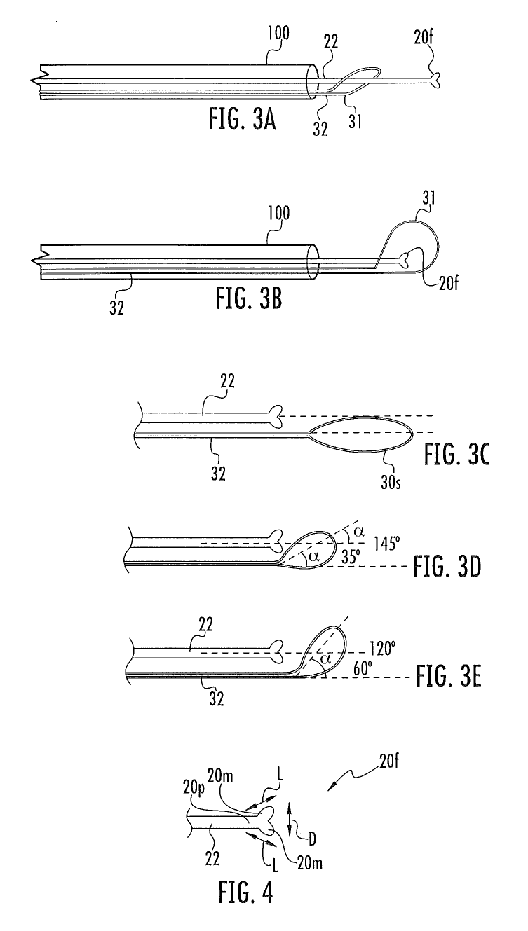

In operation, the snare loop 31 can reside about the forceps 20f. Each of these tools 20f, 30s can have independent mobility. The forceps 20f can be a straight forceps with jaws of various size. The snare 30s can also vary in size. The snare 30s can angulate so that the loop 31 can extend from the working channel 100 and open at a plurality of different orientations. For example, as shown in FIG. 3C, the loop 31 can take on a first orientation such as about 180 degrees (substantially parallel to the forceps 20f). As shown in FIG. 3D, the loop 31 can take on another second orientation of about 145 degrees (i.e., about 35 degrees off the centerline of or the axis of the shaft 30s). As shown in FIG. 3E, the loop 31 can take on a third orientation of about 120 degrees (i.e., about 60 degrees from the centerline of or the shaft axis 30s).

The forceps 20f typically extends and retracts about a substantially straight line. The forceps shaft 20s can be held in a sleeve or catheter with a guide channel that slidably engages the forceps shaft 30s to maintain a desired orientation. The loop 31 can be held in a compressed configuration inside the channel 100 and can expand upon release, typically to increase in size between about 10-200%.

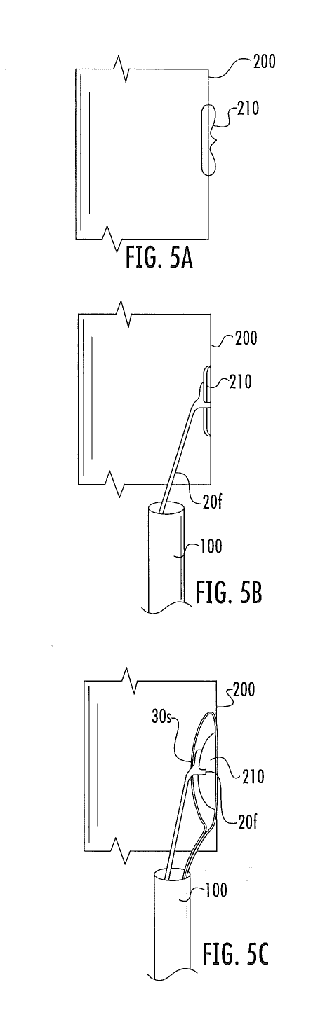

FIG. 4 illustrates an example of a forceps 20f with a pair of cooperating forcep members 20m, each having a length “L” of between about 3-10 mm, typically about 5 mm, from a pivot 20p to its forward end and being able to open a distance “D” of between about 2-15 mm, typically about 10 mm. However, the forceps 20f may be provided in other sizes and dimensions.

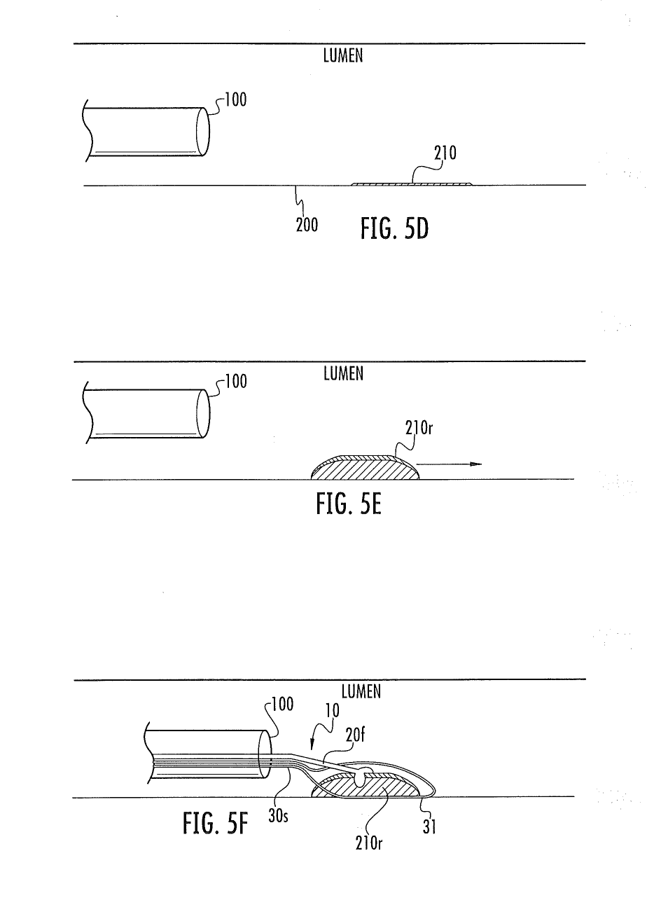

As shown in FIGS. 5A-5C, once the forceps 20f is extended out of the working channel 100 and has grasped target tissue 210 (e.g., a polyp 210 attached to a colon tissue wall 200), the snare 30s can be extended. As the loop of the snare 31 goes out of the channel 100, the snare diameter can enlarge and loop over a perimeter of the grasped target tissue 210 and the forceps 20f. The snare 30s will then close, grabbing the tissue 210 and helping in excision. FIGS. 5D-5F illustrate that the target tissue 210 (e.g., polyp) can be injected with a fluid such as saline to cause the tissue (e.g., polyp) to take on a raised contour 210r to facilitate removal by the tool 10. A needle tool can be provided as one of the tools in the same or a different working channel of the tools 20f, 30s. Optionally, the forceps 20f may also include a needle that can be selectively slid forward from a channel in the shaft 22 to inject, then retract, for the grasping. The snare 30s can be attached to a cautery cable, for cauterization if that is needed. The cooperating forceps and snare 201, 30s can ensure the tissue is grabbed easily.

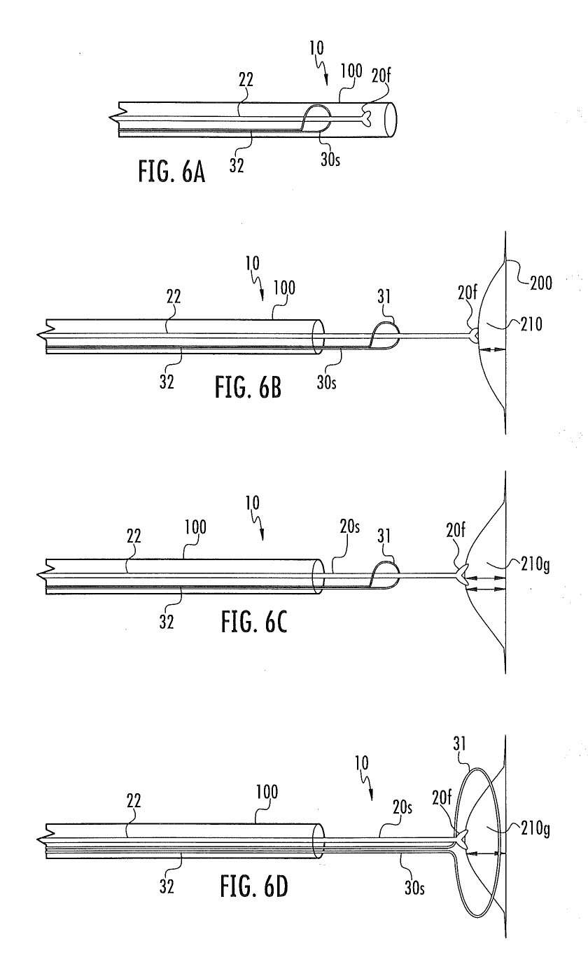

It is believed that, currently, flat polyps are difficult to grasp as the conventional snare tends to keep sliding over the tissue without grasping. FIGS. 6A-6D illustrate a series of steps that can be used to excise substantially flat polyps using the tool 10 with the forceps 20f and snare 30s. As shown, the polyp 210 can be grasped (FIG. 6B), then pulled away from the colon wall 200 by the forceps 20f (FIG. 6C) to “tent” the polyp. FIG. 6C shows that the forceps 20f can form an anchor and/or guideline about which the snare 30s can travel. In such areas, the forceps 20f can grab the polyp and anchor it so that the snare 30s can easily slide over the forceps 20f and grab the tissue. FIG. 6D illustrates that the loop 31 is then able to surround the tented polyp for ease in excision. This combination of forceps 20f and snare 30s is believed to provide a significant advantage over the current devices as the forceps 20f provide an anchor over which the snare 30s will be easily able to grasp the target tissue. This will facilitate in removing not only flat polyps, but also polyps situated in areas that are difficult to reach by conventional colonoscopy devices. The anchoring and cooperating actions of the combination tool device 10 may be particularly important in excising right sided polyps, particularly just proximal to the hepatic flexure.

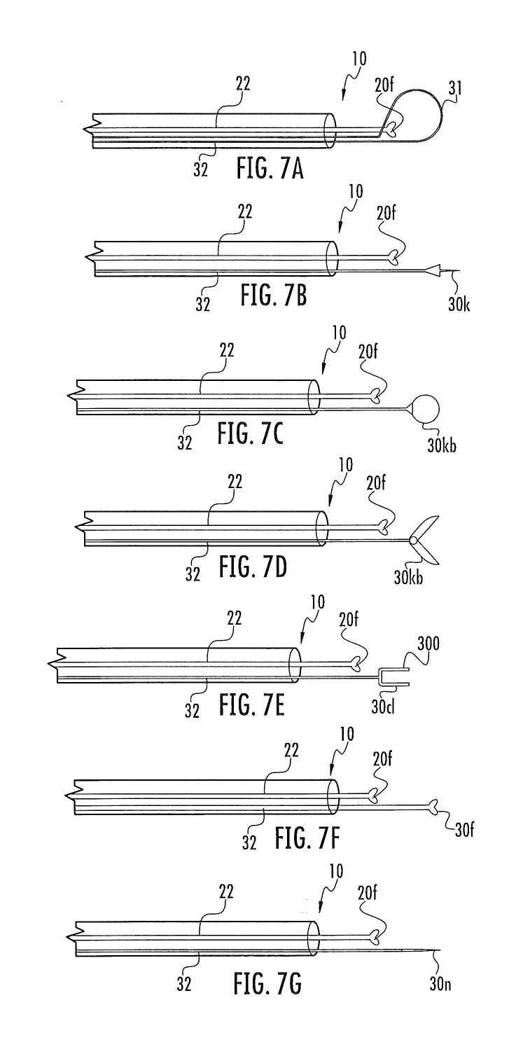

FIGS. 7A-7G are schematic illustrations of various surgical devices 10 with examples of tools 20, 30. In these figures, the device 10 includes the forceps 20f discussed above. FIG. 7A illustrates that the device 10 also includes the snare 30s. FIG. 7B illustrates that the device 10 includes the forceps 20f and a needle-knife 30k. FIG. 7C illustrates that the device 10 includes the forceps 20f and a knob 30kb. FIG. 7D illustrates that the device 10 includes the forceps 20f and scissors 30sc. FIG. 7E illustrates that the device 10 includes the forceps 20f and a clip 30c1. FIG. 7F illustrates that the device 10 includes the forceps 20f with a secondary forceps 30f. FIG. 7G illustrates that the device 10 includes the forceps 20f with a needle 30n. Examples of these devices will be discussed further below.

The forceps 20f and scissors 30s combination can be configured so that the forceps 20f and scissors 30s are housed in the same working channel 100 of the endoscope as shown in FIG. 7D. The scissors 30s can be angulated at different angles, such as those described above with respect to the snare 30s, e.g., about 180, about 145 and about 120 degrees in relation to the forceps. The scissors hands can be about 5 mm in length and can have a single hand motion (one hand moves and the other is substantially fixed). As described above with respect to the snare 30s, the forceps 20f will grab the tissue that needs dissection and the scissors can cut while the tissue is extended by the forceps.

Scissors 30s can be advanced to proceed with dissection. The scissors can also be attached to a cautery cable to use cauterization during dissection. The tissue plane can be generated by pulling the forceps that will give some triangulation. These two devices can extend out of the same channel 100 in the same plane, but at different angulations as discussed above.

With respect to the tool shown in FIG. 7C, the forceps 20f can operate as discussed above. The knob 30kb is a spherical solid or hollow end member that can be used to force blunt dissection. Once again, the knob 30kb can be attached to the cautery cable so that cautery can be used when necessary. The knob 30kb can also extend out of the working channel in different angulations as discussed above with respect to the snare and scissors, such as about 180, about 145 and about 120 degrees with relation to the forceps 20f. In some particular embodiments, the diameter of the knob 30ib can be about 3 mm (10 fr). The forceps 20f can be first pushed out (slid forward) of the channel, then can grab the tissue. Following this, the knob 30kb can be extended out. By creating traction on the tissues, the knob will be able to perform blunt dissection using cautery when needed. The main purpose of the knob is to create and expand the normally existing tissue planes within the body. This will be very useful when performing submucosal dissection as in Barrett's esophagus in removing dysplastic epithelium or, as described above, removing flat polyps. This device will also be helpful in dissection of tissue planes particularly when performing NOTES procedure.

With respect to the device 10 shown in FIG. 7G (forceps-needle), the device 10 includes a forceps 20f as described previously as well as a hollow needle 30n for injection. The needle length can be between about 3-15 mm, typically about 10 mm. The main purpose of this device is to inject a desired agent, such as saline, medicaments, or other treatments at an exact location. This will be particularly useful in NOTES procedure.

With respect to the device shown in FIG. 7E (forceps-clip), the device 10 includes a forceps 20f as described above and a clip delivery mechanism 30c with a clip 300 that is used to close a perforation. With the present technology, it is very difficult to close a large perforation as the distance between the two prongs of the open clip is only 1.0-1.5 cm. If the opening is larger than 1.5 cm, it is not possible to approximate the edges as the clip prongs usually tear through the tissue. By using a forceps-clip combination device, the forceps will grab one edge of the perforation and approximate to the opposite edge and a clip can then be applied over this perforation closing it securely. The forceps will be first pushed out and will grab one of the edges of the perforation and bring it closer to the opposite edge. The clip 300 will then be advanced and applied to the two edges of the perforation.

With respect to the embodiment shown in FIG. 7B (forceps-needle knife), the device 10 also include a forceps as described above. The other component will be a needle knife 30k. A needle knife 30k can be a monofilament metallic wire that can be brought out of an elastomeric (e.g., polymer and/or plastic) or other protective covering a distance such as between about 0.5-2 cm, typically about 1 cm. This metallic wire can be connected to the cautery device and, upon its activation, will cut the tissue. The forceps 20f will first be brought out of the device and grab the tissue and create traction. The needle knife 30k is then brought out and applied precisely at the point of the intended cut resulting in sharp dissection of the tissue.

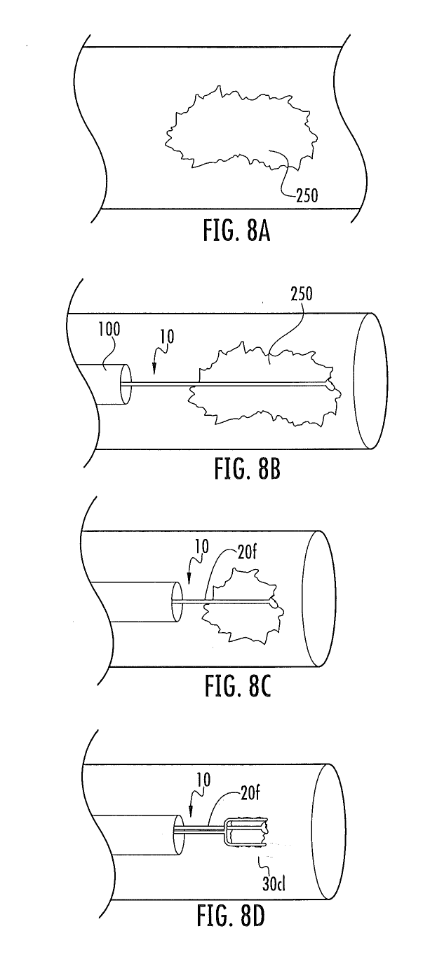

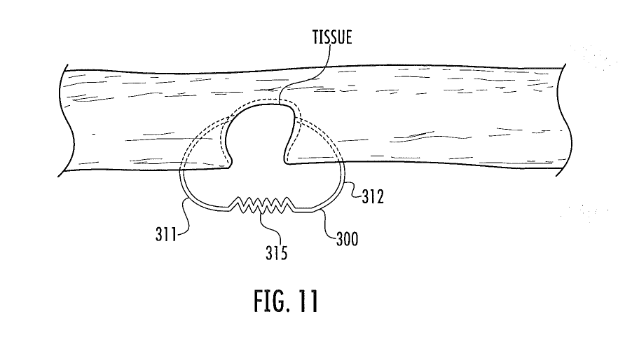

FIGS. 8A-8D show a sequence of steps that can be used to close a hole 250 such as may be associated with a perforation in a lumen using the tool shown in FIG. 7E according to embodiments of the invention. The forceps 20f can grasp a perforation edge and pull it closer to an opposing side to make the hole smaller. The clip delivery mechanism 30c1 can release the clip that then holds the edges of the hole together. The clip 300 can comprise a shape memory alloy material. In some embodiments, the clip 300 comprises at least two flexible legs 311, 312, each having a respective free end, each leg formed of shape memory material held in a substantially (straight) linear shape in the endoscope. Examples of resiliently configured (“spring”) clips 300 that have free ends 311, 312 (FIG. 11) that automatically come together at deployment from an endoscope channel to pull tissue together, which is particularly suitable for placement using an endoscope, are described in more detail in co-pending U.S. application Ser. No. 13/295,417, the contents of which are hereby incorporated by reference as if recited in full herein. Briefly stated, the clips 300 can have a flexible clip or clamp-like body that has two legs 311, 312 separated by a resilient member 315. The resilient member 315 can be described as a crown with opposing end portions, each end portion attached to one end of a respective leg 311, 312. The other ends of the legs 311, 312 are free so as to be able to contact local tissue as shown in FIG. 11. The legs 311, 312 can be formed from a shape memory material so that they can be collapsed inside an endoscope for intraluminal delivery to a closure site as will be discussed further below. The resilient member 315 can comprise a flexible elastomeric (e.g., polymeric or rubber) band, a woven or braided resilient stretchable material, a (coil) spring, a length of elastic SMP, or other resilient member, or combinations of different types of resilient members. The legs 311, 312 and the resilient member 315 can be relatively thin. The legs 311, 312 can have a diameter or cross-sectional width/height of between about 1 mm to about mm with typical lengths being between about 10-30 mm. In some embodiments, the clip 300 can be used for tissue closures for dissections, perforations and the like having sizes of between about 10 mm to about 25 mm. The resilient member 315 can have a cross-sectional width/height (e.g., diameter) that is between about 0.1 mm to about 5 mm and have sufficient resiliency or elasticity to be able to pull the legs 311, 312 together to close the target opening, perforation or defect. In some embodiments, the resilient member 315 has a length that is between about 20% to about 50% the length of a respective leg or prong 311, 312.

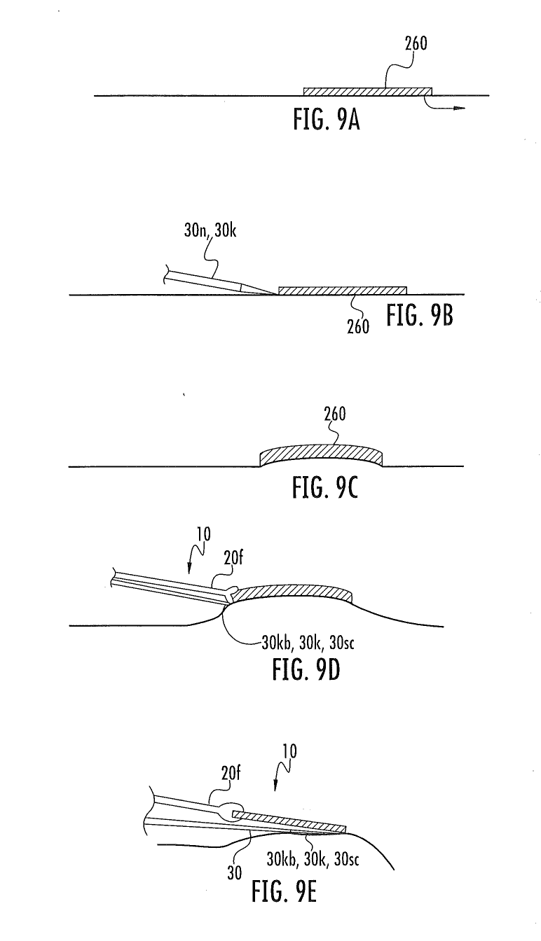

FIGS. 9A-9E illustrate an example of a submucosal dissection. FIG. 9A illustrates a flat lesion that is to be dissected out of a patient. FIG. 9B illustrates that an injection of fluid into the submucosal plane can be used to raise the flat lesion and FIG. 9C, illustrates the flat lesion 260 raised above submucosal edema. The needle used to inject the fluid can be part of the medical device 10 (e.g., 30n, 30k, or integrated into the forceps) or may be a separate tool. The device 10 can be used to perform the dissection. As above, the forceps 20f can pull the flat lesion away from the wall while the other tool, e.g., 30kb, 30k, 30sc performs the dissection.

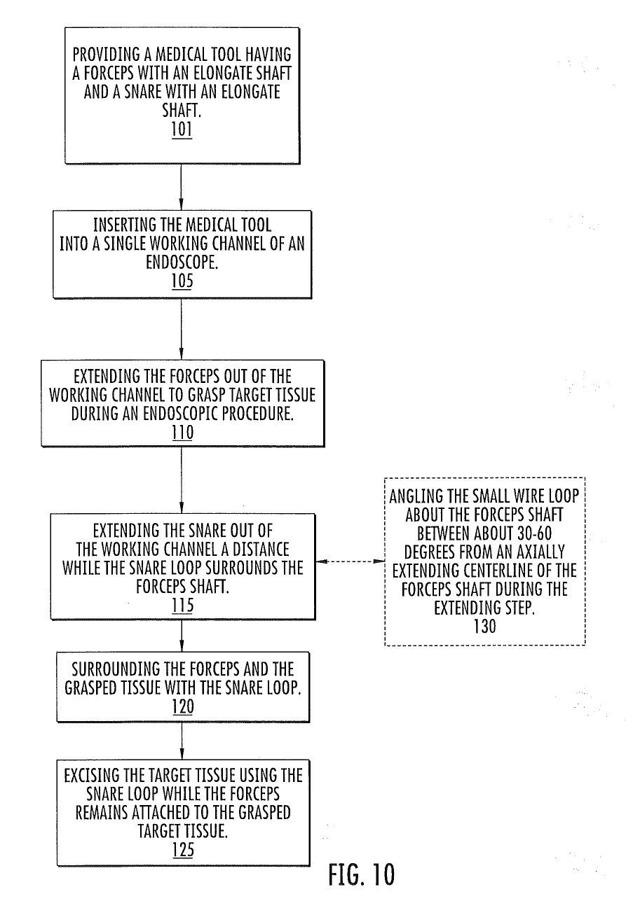

FIG. 10 is a flow chart of exemplary steps that can be used to carry out methods according to embodiments of the present invention. A medical tool having a forceps with an elongate shaft and a snare with an elongate shaft is provided (block 101). The medical tool is inserted into a single working channel of an endoscope (block 105). The forceps is extended out of the working channel to grasp target tissue of a patient during an endoscopic procedure (block 110). Then the snare is extended out of the working channel a distance while the snare loop surrounds the forceps shaft (block 115). The forceps and the grasped tissue are then surrounded by the snare loop (block 120). The target tissue is excised using the snare loop while the forceps remains attached to the grasped target tissue (block 125). Optionally, the wire loop can be angled at different orientations about the forceps shaft between about 30-60 degrees from an axially extending centerline of the forceps shaft during the extending step (block 130).

The devices and tools are particularly suitable for endoscopic procedures and more particularly, NOTES procedures.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. In the claims, means-plus-function clauses, if used, are intended to cover the structures described herein as performing the recited function and not only structural equivalents but also equivalent structures. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

That which is claimed is:

1. An endoscopic medical device for removing tissue, comprising:
a forceps with an elongate shaft slidably residing in a first working channel of an endoscope and having an externally accessible forceps control; and
a snare with an elongate shaft comprising a wire loop slidably residing in the first working channel of the endoscope, the snare having an externally accessible snare control,
wherein, the forceps and snare shafts reside adjacent to and substantially parallel to each other inside the first working channel of the endoscope, one above or to the side of the other,
and wherein, in use, the forceps and snare cooperate so that the forceps control directs the forceps to extend out of the endoscopic first working channel and grasp target tissue, then the snare control directs the snare loop to exit the working channel while the snare loop encircles the forceps shaft and extends a distance sufficient to surround the forceps and grasped tissue so that the forceps and the snare loop cooperate while the snare excises the grasped target tissue.

2. The medical device of claim 1, wherein the snare loop is a closed loop that resides about the forceps shaft inside the working channel of the endoscope prior to extension from the working channel.

3. The medical device of claim 1, wherein the snare loop is able to angulate with respect to the forceps to be able to take on different angles relative to the forceps.

4. The medical device of claim 3, wherein the snare loop is able to move from an orientation that is substantially parallel to the forceps to an orientation that is between 30-60 degrees relative to an longitudinally extending centerline of the forceps.

5. The medical device of claim 1, wherein the loop is configured to enlarge upon exiting the working channel.

6. A method of excising target tissue from a patient, comprising:
providing a medical tool having a forceps with an elongate shaft and a snare with an elongate shaft;
inserting the medical tool into a single working channel of an endoscope;
extending the forceps out of the working channel to grasp target tissue during an endoscopic procedure of a patient; then extending the snare out of the working channel a distance while the snare loop surrounds the forceps shaft; then
surrounding the forceps and the grasped tissue with the snare loop; and
excising the target tissue using the snare loop while the forceps remains attached to the grasped target tissue.

7. The method of claim 6, further comprising angling the wire loop about the forceps shaft between about 30-60 degrees from an axially extending centerline of the forceps shaft during the extending step.

8. The method of claim 6, wherein the snare loop is configured to enlarge upon exiting the working channel.

9. The method of claim 6, wherein the target tissue is a polyp.

10. An endoscopic tool, comprising:
a forceps tool with an elongate shaft; and
a second tool with an elongate shaft,
wherein, each tool is configured to reside inside a single working channel of an endoscope.

11. The tool of claim 10, wherein the second tool is a clip delivery device holding a deployable clip that attaches to local tissue.

12. The tool of claim 10, wherein the second tool is at least one of a knob, scissors, knife, needle-knife, secondary forceps or needle.

13. The tool of claim 10, wherein the forceps and second tool cooperate for submucosal dissection during a NOTES procedure.

14. A method of removing polyps or performing a submucosal dissection, comprising:
providing a medical device with a forceps and a second cooperating tool, both adapted to reside together in a single working channel of an endoscope;
extending the forceps out of the working channel to grasp target tissue; then
using the second tool to carry out the submucosal dissection of the grasped tissue.

15. The method of claim 14, wherein the second tool comprises a wire snare.

16. The method of claim 14, wherein the second tool is at least one of a knife, a needle, a needle-knife or a knob.

17. A method of repairing a perforation during an endoscopic procedure, comprising:
providing a medical device with a forceps and a second cooperating tool releasably holding a clip, both tools adapted to reside together in a single working channel of an endoscope;
extending the forceps out of the working channel to grasp target tissue to pull the target tissue to make a perforation smaller; then
using the second tool to attach the clip to close the perforation while the forceps holds the grasped tissue.
